(19) United States
(12) Patent Application Publication
(10) Pub. No.: US 2026/0115246 A1
(43) Pub. Date: Apr. 30, 2026

(54) TRADITIONAL CHINESE MEDICINE COMPOSITION FOR TREATMENT OF LUNG TUMOR, PREPARATION METHOD THEREFOR, AND USE THEREOF

(71) Applicant: ANHUA HENGTAI (BEIJING) PHARMACEUTICAL TECHNOLOGY CO., LTD, BEIJING (CN)

(72) Inventor: ANYI ZUO, BEIJING (CN)

(21) Appl. No.: 18/988,777

(22) Filed: Dec. 19, 2024

(30) Foreign Application Priority Data
Oct. 24, 2024 (CN) 202411323157.8

(51) Int. Cl. A61K 36/284; A61K 36/074; A61K 36/076; A61K 36/236; A61K 36/285; A61K 36/287; A61K 36/355; A61K 36/481; A61K 36/484; A61K 36/534; A61K 36/54; A61K 36/61; A61P 35/00

(52) U.S. Cl. CPC A61K 36/284; A61K 36/074; A61K 36/076; A61K 36/236; A61K 36/285; A61K 36/287; A61K 36/355; A61K 36/481; A61K 36/484; A61K 36/534; A61K 36/54; A61K 36/61; A61P 35/00; A61K 2236/15; A61K 2236/331; A61K 2236/333; A61K 2236/51; A61K 2236/53

(57) ABSTRACT

A traditional Chinese medicine composition for treatment of lung tumor, a preparation method therefor, and a use thereof are provided. The traditional Chinese medicine composition includes the following raw materials by weight: 5-15 parts of Dendranthema morifolium, 2-12 parts of Lonicera japonica, 5-15 parts of Mentha haplocalyx, 5-15 parts of Wolfiporia cocos, 10-20 parts of Atractylodes lancea, 0.5-3 parts of Cinnamomum cassia, 0.5-3 parts of Syzygium aromaticum, 3-12 parts of Astragalus membranaceus, 5-15 parts of Ganoderma lucidum, 10-20 parts of Ligusticum chuanxiong, 5-12 parts of Aucklandia radix, and 2-10 parts of Glycyrrhiza uralensis.

### CROSS-REFERENCE TO RELATED PATENT APPLICATION

This application claims priority to Chinese Patent Application No. 202411323157.8, filed on Oct. 24, 2024, the content of all of which is incorporated herein by reference.

### FIELD OF THE DISCLOSURE

The present disclosure relates to the technical field of traditional Chinese medicine, in particular to a traditional Chinese medicine composition for treatment of lung tumor, a preparation method therefor, and a use thereof.

### BACKGROUND OF THE DISCLOSURE

Cancer in a human body is generally caused by imbalance of immune function and uncontrolled growth of cancer cell due to a weakened immune system of the human body.

Immunotherapy is a new treatment method for cancer in recent years. Immunotherapy in Western medicine is usually combined with chemotherapy and radiotherapy, which has certain therapeutic effects, but there is a technical problem that immunotherapy has side effects and drug tolerance may develop.

Chinese Patent Application No. 201710147162.1 (Publication Number CN108567820B) discloses a traditional Chinese medicine composition for treatment of lung tumor, which solves the above-mentioned technical problem through combination of thirteen medicinal herbs, but an inhibition effect of this traditional Chinese medicine composition on lung tumor growth still needs to be improved.

### SUMMARY OF THE DISCLOSURE

According to the above-mentioned defects of the prior art, a purpose of the present disclosure is to provide a traditional Chinese medicine composition for treatment of lung tumor, a preparation method therefor, and a use thereof, which has a significant inhibition effect on tumor growth.

To achieve the above-mentioned purpose, a technical solution adopted by the present disclosure is: a traditional Chinese medicine composition for treatment of lung tumor is provided, including the following raw materials by weight: 5-15 parts of Dendranthema morifolium, 2-12 parts of Lonicera japonica, 5-15 parts of Mentha haplocalyx, 5-15 parts of Wolfiporia cocos, 10-20 parts of Atractylodes lancea, 0.5-3 parts of Cinnamomum cassia, 0.5-3 parts of Syzygium aromaticum, 3-12 parts of Astragalus membranaceus, 5-15 parts of Ganoderma lucidum, 10-20 parts of Ligusticum chuanxiong, 5-12 parts of Aucklandia radix, and 2-10 parts of Glycyrrhiza uralensis.

In some embodiments, the traditional Chinese medicine composition for treatment of lung tumor includes the following raw materials by weight: 5-10 parts of Dendranthema morifolium, 3-8 parts of Lonicera japonica, 5-10 parts of Mentha haplocalyx, 5-10 parts of Wolfiporia cocos, 12-20 parts of Atractylodes lancea, 0.5-3 parts of Cinnamomum cassia, 0.5-3 parts of Syzygium aromaticum, 5-10 parts of Astragalus membranaceus, 5-10 parts of Ganoderma lucidum, 11-15 parts of Ligusticum chuanxiong, 8-12 parts of Aucklandia radix, and 3-8 parts of Glycyrrhiza uralensis.

In some embodiments, the traditional Chinese medicine composition for treatment of lung tumor includes the following raw materials by weight: 6-9 parts of Dendranthema morifolium, 3-6 parts of Lonicera japonica, 6-9 parts of Mentha haplocalyx, 6-9 parts of Wolfiporia cocos, 15-18 parts of Atractylodes lancea, 1-3 parts of Cinnamomum cassia, 0.5-1.5 parts of Syzygium aromaticum, 6-10 parts of Astragalus membranaceus, 6-10 parts of Ganoderma lucidum, 12-15 parts of Ligusticum chuanxiong, 8-11 parts of Aucklandia radix, and 3-6 parts of Glycyrrhiza uralensis.

In some embodiments, a dosage form of the traditional Chinese medicine composition for treatment of lung tumor is injection, honey-refined pill, hydrous pill, capsule, tablet, dripping pill, powder, oral liquid medicine, gel, extract, or film.

To achieve the above-mentioned purpose, a technical solution adopted by the present disclosure is: a preparation method for the traditional Chinese medicine composition for treatment of lung tumor, including the following steps:
(1) weighing each of tradition Chinese medicine raw materials in accordance with the above weight ratio, separately grinding the raw materials to a particle size of 10-100 mesh, sieving, and then mixing evenly to obtain an evenly-mixed traditional Chinese medicine mixture;
(2) preparing an ethanol solution with a concentration of 40-95% for later use, soaking the evenly-mixed traditional Chinese medicine mixture in the prepared ethanol solution with the concentration of 40-95% for 10-48 hours to obtain a soaked traditional Chinese medicine mixture, adding the soaked traditional Chinese medicine mixture into a percolating bucket in batches, and then gently pressing the soaked traditional Chinese medicine mixture to a flat state;
(3) adding the prepared ethanol solution with the concentration of 40-95% into the percolating bucket until the ethanol solution is 1-2 cm higher than a surface of the soaked traditional Chinese medicine mixture, soaking for a preset duration, then adding remaining ethanol solution into the percolating bucket for percolation to obtain an alcohol solution and medicine herb residue, keeping the medicine herb residue for later use, and concentrating the obtained alcohol solution under reduced pressure and then drying to obtain a traditional Chinese medicine composite powder A; and,
(4) adding water to the medicine herb residue, wherein the amount of the water is 2-10 times the amount of the medicine herb residue, decocting with water and extracting for 0.5-2 hours, centrifuging and filtering to obtain a medicine solution, concentrating the medicine solution under reduced pressure and then drying to obtain a traditional Chinese medicine composite powder B, and mixing the traditional Chinese medicine composite powder A and the traditional Chinese medicine composite powder B evenly to obtain the traditional Chinese medicine composition.

In some embodiments, in step (1), each of the tradition Chinese medicine raw materials are separately grinded to the particle size of 20-40 mesh; in step (2) and step (3), the concentration of the prepared ethanol solution is 60-80%.

In some embodiments, in step (2), the evenly-mixed traditional Chinese medicine mixture is soaked for 18-30 hours; in step (3), the preset duration is 2-48 hours.

To achieve the above-mentioned purpose, a technical solution adopted by the present disclosure is: a preparation method for the traditional Chinese medicine composition for treatment of lung tumor, including the following steps:
(1) weighing medicinal herbs of Ligusticum chuanxiong, Atractylodes lancea, Aucklandia radix, Syzygium aromaticum, Cinnamomum cassia, and Mentha haplocalyx in accordance with the above weight ratio, adding water in moderation to extract volatile oil, adding cyclodextrin for inclusion, and decocting with water and then filtering to obtain filtrate for later use;
(2) mixing medicinal herb residue obtained after extracting the volatile oil with weighed Wolfiporia cocos, Astragalus membranaceus, Dendranthema morifolium, Ganoderma lucidum, Lonicera japonica, and Glycyrrhiza uralensis in accordance with the above weight ratio, adding water for decocting, filtering to obtain filtered medicinal herb solution, and combining the filtered medicinal herb solution with the filtrate to obtain a mixed medicine herb solution;
(3) concentrating the mixed medicine herb solution through a water bath to obtain a clear paste, adding ethanol to increase an alcohol content to 50-80% to obtain a medicine alcohol mixture; and,
(4) concentrating the medicine alcohol mixture under reduced pressure, drying, and then adding volatile-oil cyclodextrin inclusion complex to obtain the traditional Chinese medicine composition.

In some embodiments, in step (1), a volatile-oil extraction solvent is water, the amount of the volatile-oil extraction solvent is 4-15 times the amount the medicinal herbs, an extraction duration for extracting the volatile oil is 2-12 hours, the obtained volatile oil is performed cyclodextrin inclusion at 40° C. or below, and an inclusion duration is 1-10 hours.

To achieve the above-mentioned purpose, a technical solution adopted by the present disclosure is: a use of a traditional Chinese medicine composition, and the traditional Chinese medicine composition is combined with cyclophosphamide for treatment of lung tumor.

### Beneficial Effects of the Present Disclosure

1. In the present disclosure, Ganoderma lucidum tonifies heart, liver, spleen, lung, and kidney, invigorates spleen and replenishes Qi, and enhances body immunity; Astragalus membranaceus invigorates spleen and nourishes lung, benefits qi for ascending Yang energy, and enhances body immunity; Ganoderma lucidum and Astragalus membranaceus can inhibit growth of tumor telomerase; Atractylodes lancea, Wolfiporia cocos, and Glycyrrhiza uralensis invigorate spleen and eliminate dampness; Cinnamomum cassia, Aucklandia radix, Syzygium aromaticum are pungent and warm, and can regulate Qi, warm spleen and stomach, dispel coldness, and relieve pain; Lonicera japonica clears heat-toxin; Dendranthema morifolium clears liver, improves eyesight and removes toxicity; Mentha haplocalyx is pungent and cold and removes toxicity, disperses stagnated liver Qi for relieving Qi stagnation; Ligusticum chuanxiong regulates Qi, promotes blood circulation, and relieves pain. Ganoderma lucidum and Astragalus membranaceus are as King material to invigorate spleen and replenish Qi, tonify heart, liver, spleen, lung, and kidney, strengthen body resistance and consolidate body vitality, and resist tumor. Atractylodes lancea, Wolfiporia cocos, and Glycyrrhiza uralensis are as Minister material to invigorate spleen and eliminate dampness, and Cinnamomum cassia, Aucklandia radix, and Syzygium aromaticum are as Minister material to regulate Qi and warm spleen and stomach, dispel coldness and relieve pain. Dendranthema morifolium and Lonicera japonica are as Assistant material to clear heat-toxin. Ligusticum chuanxiong and Mentha haplocalyx are as Messenger material to clear liver, promote blood circulation, and relieve pain.
2. The traditional Chinese medicine composition of the present disclosure has a significant inhibition effect on tumor growth of A-549 tumor-bearing nude mouse.
3. The traditional Chinese medicine composition of the present disclosure can increase the numbers of white blood cells, neutrophils, lymphocytes, and monocytes when used alone or in combination with cyclosphamide.

### BRIEF DESCRIPTION OF THE DRAWINGS

In order to more clearly illustrate the technical solutions in the embodiments of the present disclosure, the drawings needed to be used in the embodiments of the present disclosure are briefly introduced below. Obviously, the drawings in the following description are only some embodiments of the present disclosure. For those ordinary skilled in the art, other drawings can be obtained based on the present drawings without creative efforts.

FIG. 1 is a tumor growth curve in the present disclosure.

FIG. 2 is another tumor growth curve in the present disclosure.

FIG. 3 is a change curve of body weight in the present disclosure.

FIG. 4 is a change curve of rate of rise of body weight in the present disclosure.

FIG. 5 is a photograph of tumor tissues in each group in the present disclosure.

### DETAILED DESCRIPTION OF THE EXEMPLARY EMBODIMENTS

The present disclosure is described in further detail below in conjunction with the accompanying drawings.

The present embodiments are only an explanation of the present disclosure and do not limit the present disclosure. After reading the present specification, those skilled in the art may make modifications to the present embodiments as needed without any creative work, and the modifications are within the protection scope of the claims of the present disclosure.

### Embodiment 1

The present disclosure provides a traditional Chinese medicine composition for treatment of lung tumor, which includes following raw materials with a weight ratio of: 9% of Dendranthema morifolium (Ramat.) Tzvel., 5% of Lonicera japonica Thunb., 7% of Mentha haplocalyx Briq., 8% of Wolfiporia cocos (Schw.) Ryv. & Gibn., 16% of Atractylodes lancea (Thunb.) DC., 2% of Cinnamomum cassia Presl., 1% of Syzygium aromaticum, 10% of Astragalus membranaceus, 12% of Ganoderma lucidum (Leyss. ex Fr.) Karst., 13% of Ligusticum chuanxiong, 9% of Aucklandia radix, and 8% of Glycyrrhiza uralensis Fisch.

A preparation method for the traditional Chinese medicine composition for treatment of lung tumor includes steps of: weighing each of the above-mentioned traditional Chinese medicine raw materials according to the above-mentioned weight ratio, and separately grinding to a particle size of 20 mesh, sieving, and mixing evenly; preparing an ethanol solution with a concentration of 70% for later use; soaking evenly-mixed traditional Chinese medicine mixture in the prepared 70% ethanol solution for at least 24 hours, adding soaked traditional Chinese medicine mixture into a percolating bucket in batches, and gently pressing to a flat state; adding the prepared 70% ethanol solution 1-2 cm to be higher than a surface of medicinal herbs, soaking for 18-30 hours (preferably 20 hours), then adding remaining ethanol solution into the percolating bucket until percolation occurs, and keeping medicine herb residue for later use; concentrating obtained solution under reduced pressure, drying to obtain a traditional Chinese medicine composite powder A; adding water to the medicine herb residue, where the amount of the water is 2-10 times (preferably 8 times) the amount of the medicine herb residue, decocting with water and extracting for 0.5-2 hours (preferably 1 hour), centrifuging and filtering to obtain a medicine solution, and concentrating the medicine solution under reduced pressure and drying to obtain a traditional Chinese medicine composite powder B; and, mixing the traditional Chinese medicine composite powder A and the traditional Chinese medicine composite powder B evenly to obtain the traditional Chinese medicine composition.

### Embodiment 2

The present disclosure provides a traditional Chinese medicine composition for treatment of lung tumor, which includes following raw materials with a following weight ratio of: 8% of Dendranthema morifolium (Ramat.) Tzvel., 7% of Lonicera japonica Thunb., 7% of Mentha haplocalyx Briq., 9% of Wolfiporia cocos (Schw.) Ryv. & Gibn., 15% of Atractylodes lancea (Thunb.) DC., 2% of Cinnamomum cassia Presl., 2% of Syzygium aromaticum, 11% of Astragalus membranaceus, 10% of Ganoderma lucidum (Leyss. ex Fr.) Karst., 14% of Ligusticum chuanxiong, 10% of Aucklandia radix, and 5% of Glycyrrhiza uralensis Fisch.

A preparation method for the traditional Chinese medicine composition for treatment of lung tumor includes steps of: weighing medicinal herbs of Ligusticum chuanxiong, Atractylodes lancea, Aucklandia radix, Syzygium aromaticum, Cinnamomum cassia, and Mentha haplocalyx according to the above-mentioned weight ratio, and adding a volatile-oil extraction solvent in moderation to extract volatile oil, where the volatile-oil extraction solvent is water and 6 times the amount of the medicinal herbs, and an extraction duration is 2-12 hours (preferably 10 hours); after extracting the volatile oil, performing cyclodextrin inclusion at 40° C. or below for 6 hours; decocting with water and filtering to obtain filtrate for later use; mixing medicinal herb residue obtained after extracting the volatile oil with medicinal herbs of Wolfiporia cocos, Astragalus membranaceus, Dendranthema morifolium, Ganoderma lucidum, Lonicera japonica, and Glycyrrhiza uralensis weighed according to the weight ratio, and adding water for decocting 2-5 times (preferably 3 times), where each time duration is 1-2 hours (preferably 1.5 hours), and the amount of the water is 10-15 times (preferably 12 times) the amount of the medicinal herbs; filtering decocted medicinal herb solution, and combining filtered medicinal herb solution with the filtrate obtained from extracting the volatile oil to obtain a mixed medicine herb solution; concentrating the obtained mixed medicine herb solution through a water bath to obtain a clear paste with a relative density of 1.06-1.08, adding ethanol to increase an alcohol content to 60%, and standing for not less than 12 hours (preferably 48 hours) to obtain a medicine alcohol mixture; and, concentrating the medicine alcohol mixture under reduced pressure, drying, and then adding volatile-oil cyclodextrin inclusion complex to obtain the traditional Chinese medicine composition.

### Embodiment 3

The present disclosure provides a traditional Chinese medicine composition for treatment of lung tumor, which includes following raw materials with a weight ratio of: 7% of Dendranthema morifolium (Ramat.) Tzvel., 6% of Lonicera japonica Thunb., 8% of Mentha haplocalyx Briq., 11% of Wolfiporia cocos (Schw.) Ryv. & Gibn., 17% of Atractylodes lancea (Thunb.) DC., 1.5% of Cinnamomum cassia Presl., 0.5% of Syzygium aromaticum, 9% of Astragalus membranaceus, 9% of Ganoderma lucidum (Leyss. ex Fr.) Karst., 16% of Ligusticum chuanxiong, 9% of Aucklandia radix, and 6% of Glycyrrhiza uralensis Fisch.

A preparation method for the traditional Chinese medicine composition for treatment of lung tumor includes steps of: weighing each of the above-mentioned Chinese medicine raw materials according to the above-mentioned weight ratio; preparing an ethanol solution with a concentration of 60% for later use; adding the prepared ethanol solution to evenly-mixed traditional Chinese medicine mixture, where the amount of the ethanol solution is 6 times the amount of the evenly mixed traditional Chinese medicine mixture, heating, and performing reflux extraction two times (heating to slight boiling to perform the reflux extraction), where each time duration is 1-2 hours (preferably 1.5 hours); and, combining first-time extraction solution and second-time extraction, recycling ethanol under reduced pressure, and concentrating and drying to obtain the traditional Chinese medicine composition.

### Embodiment 4

The present disclosure provides a traditional Chinese medicine composition for treatment of lung tumor, which includes following raw materials with a weight ratio of: 9% of Dendranthema morifolium (Ramat.) Tzvel., 7% of Lonicera japonica Thunb., 7% of Mentha haplocalyx Briq., 8% of Wolfiporia cocos (Schw.) Ryv. & Gibn., 13% of Atractylodes lancea (Thunb.) DC., 2% of Cinnamomum cassia Presl., 1% of Syzygium aromaticum, 10% of Astragalus membranaceus, 15% of Ganoderma lucidum (Leyss. ex Fr.) Karst., 13% of Ligusticum chuanxiong, 7% of Aucklandia radix, and 8% of Glycyrrhiza uralensis Fisch.

A preparation method for the traditional Chinese medicine composition for treatment of lung tumor includes steps of: weighing each of the above-mentioned Chinese medicine raw materials according to the above-mentioned weight ratio, separately grinding to a particle size of 30 mesh, sieving, and mixing evenly; preparing an ethanol solution with a concentration of 65% for later use; soaking evenly-mixed traditional Chinese medicine mixture in the prepared 65% ethanol solution for at least 36 hours, adding soaked traditional Chinese medicine mixture into a percolating bucket in batches, and gently pressing to a flat state; adding the prepared 65% ethanol solution to be 1-2 cm higher than a surface of medicinal herbs, soaking for 18 hours, then percolating remaining ethanol solution, and keeping medicinal herb residue for later use; concentrating obtained solution under reduced pressure, drying to obtain a traditional Chinese medicine composite powder A; adding water to the medicinal herb residue, where the amount of the water is 5 times the amount of the medicinal herb residue, decocting with water and extracting for 1-2 hours (preferably 1.5 hours), centrifuging and filtering to obtain a medicine solution, and concentrating the medicine solution under reduced pressure and drying to obtain a traditional Chinese medicine composite powder B; and, mixing the traditional Chinese medicine composite powder A and the traditional Chinese medicine composite powder B evenly to obtain the traditional Chinese medicine composition.

### Embodiment 5

The present disclosure provides a traditional Chinese medicine composition for health care, which includes following raw materials with a weight ratio of: 8% of Dendranthema morifolium (Ramat.) Tzvel., 3% of Lonicera japonica Thunb., 9% of Mentha haplocalyx Briq., 9% of Wolfiporia cocos (Schw.) Ryv. & Gibn., 16.5% of Atractylodes lancea (Thunb.) DC., 1% of Cinnamomum cassia Presl., 1% of Syzygium aromaticum, 9% of Astragalus membranaceus, 9% of Ganoderma lucidum (Leyss. ex Fr.) Karst., 13.5% of Ligusticum chuanxiong, 10% of Aucklandia radix, and 11% of Glycyrrhiza uralensis Fisch.

A preparation method for the traditional Chinese medicine composition for treatment of lung tumor includes steps of: weighing medicinal herbs of Ligusticum chuanxiong, Atractylodes lancea, Aucklandia radix, Syzygium aromaticum, Cinnamomum cassia, and Mentha haplocalyx according to the above-mentioned weight ratio, and adding a volatile-oil extraction solvent in moderation to extract volatile oil, where the volatile-oil extraction solvent is water and 4 times the amount of the medicinal herbs, and a extraction duration is 2-12 hours (preferably 10 hours); after extracting the volatile oil, performing cyclodextrin inclusion at 40° C. or below for 5 hours; decocting with water and filtering to obtain filtrate for later use; mixing medicinal herb residue obtained after extracting the volatile oil with medicinal herbs of Wolfiporia cocos, Astragalus membranaceus, Dendranthema morifolium, Ganoderma lucidum, Lonicera japonica, and Glycyrrhiza uralensis weighed according to the weight ratio, and adding water for decocting 2-5 times (preferably 3 times), where each time duration is 1-2 hours (preferably 1.5 hours), and the amount of the water is 10-15 times (preferably 12 times) the amount of the medicinal herbs; filtering decocted medicinal herb solution, and combining filtered medicinal herb solution with the filtrate obtained from extracting the volatile oil to obtain a mixed medicine herb solution; concentrating the obtained mixed medicine herb solution through a water bath to obtain a clear paste with a relative density of 1.06-1.08, adding ethanol to increase an alcohol content to 60%, and standing for not less than 12 hours (preferably 24 hours) to obtain a medicine alcohol mixture; and, concentrating the medicine alcohol mixture under reduced pressure, drying, and then adding volatile-oil cyclodextrin inclusion complex to obtain the traditional Chinese medicine composition.

### Pharmacodynamic Test:

In order to verify efficacy of the present disclosure in tumor treatment, the traditional Chinese medicine composition prepared by the preparation method in Embodiment 1 was used for the following efficacy test.

### 1. Test Material

### 1.1. Test Medicine

A traditional Chinese medicine composition containing Sophora flavescens Aiton. (hereinafter referred to as an original prescription) and the traditional Chinese medicine composition prepared by the preparation method in Embodiment 1 (hereinafter referred to as the present prescription) both had the same physical appearance of black-brown viscous liquid, the same usage of oral administration, the same sample drug content of 1 g crude drug/ml, the same storage condition of refrigerated storage, and the same attention of heating medicine liquid to 35-40° C. and shaking evenly before use. A recommended clinical dosage for the original prescription was 7.6 g crude drug/60 kg/d, and a recommended clinical dosage for the present prescription was 7.0 g crude drug/60 kg/d.

### 1.2. Active Control Medicine

Cyclophosphamide for injection, was produced by Jiangsu Shengdi Pharmaceutical Co., Ltd., with an indication that cyclophosphamide is a widely used anti-cancer drug having good therapeutic effects on malignant lymphoma, acute or chronic lymphocytic leukemia, multiple myeloma, and had certain therapeutic effects on lung cancer and breast cancer. The batch number is 17020625, the production date is Feb. 6, 2017, and the expiration date is Feb. 5, 2019. The ingredient is cyclophosphamide, the physical appearance is white crystalline or crystalline powder. The drug specification is 0.2 g/bottle and 10 bottles/box. The usage and dosage of intravenous injection for adults, 500-1000 mg/m² per dose according to body surface area (BSA), one dose a week, two doses in a row, and resting for 1-2 weeks and then repeating. The storage condition is shading and sealing, and stored at 30° C. or below.

### 1.3. Test Animal

| | | Body | | | License | Certification | |
|---|---|---|---|---|---|---|---|
| Strain | Level | Weight | Quantity | Sex | Number | Number | Source |
| BALB/c- | Specific | 18-20 | 88 | Half | SCXK | 11400700223720 | Beijing |
| nu | Pathogen | g | | male | (Beijing) | | Weitonglihua |
| mice | Free | | | and | 2016- | | Experimental |
| | (SPF) | | | half | 0011 | | Animal |
| | | | | female | | | Technology |
| | | | | | | | Co., Ltd |

### 1.4. Tumor Cell Line

| Cell Name | Use | Source |
|---|---|---|
| A549 human lung | Preparing nude | Cell Resource Center, |
| adenocarcinoma | mouse lung cancer | Institute of Basic Medical |
| passage | xenograft model | Sciences, Chinese |
| cell line | | Academy of Medical |
| | | Sciences |

### 1.5. Test Reagent

| Name | Manufacturer | Batch Number | Expiration date | Use |
|---|---|---|---|---|
| Trypsin-EDTA | Gibco | 1798279 | 2018 May | Digesting cell |
| DMEM (high | Gibco | 8117062 | 2018 March | Resuspending |
| glucose) | | | | cell |
| Sterile saline | Shijiazhuang | 1602153204 | 2018 Feb. 14 | Tumor |
| solution | Siyao Co., Ltd | | | homogenate |

### 1.6. Test Apparatus

| Apparatus Name | Brand | Model | Use |
|---|---|---|---|
| Clean bench | AIRTECH, USA | SW-CJ-2FD | Sterile operation |
| CO₂ incubator | Thermo Scientific, | Thermo-371 | Cell culture |
| | USA | | |
| Inverted | Olympus, Japan | OLYMPUS IX71 | Cell observation |
| microscope | | | |
| Full-automatic cell | CountStar | IC1000 | Cell count |
| counter | | | |
| Centrifuge | Eppendorf, Germany | Centrifuge 5430 | Cell centrifugation |
| High-speed | Eppendorf, Germany | 5810R | Blood |
| centrifuge | | | centrifugation |
| Refrigerator | BSH Home | BCD-254 | Storing reagent |
| | Appliance Co., Ltd | | and drug |
| High-temperature | ALP | CLG-32L | Disinfecting test |
| steam sterilizer | | | article |
| Electrothermal | ANKE | 101A-2E | Drying test article |
| blast drying oven | | | after high pressure |
| Digital vernier | Shanghai Hengsheng | METR-ISO | Measuring length |
| caliper | Tool Co., Ltd | | and width of tumor |
| Electronic balance | OHAUS Instrument | SE202F | Sterilely weighing |
| | (Shanghai) Co., Ltd | | tumor |
| Electronic balance | Sartorius | BSA3202S-CW | Weighing body |
| | | | weight of nude |
| | | | mouse |
| Electronic balance | METTLERTOLEDO | AL204 | Weighing visceral |
| | | | organ and tumor |

### 1.7. Main Test Consumable

| Name | Specification | |
|---|---|---|
| Plastic culture flask | 225 | cm² |
| Pipette tip | Yellow (200 μl), blue (1000 μl) | |
| Centrifuge tube | 1 ml, 5 ml | |
| Cell Strainer | 70 | μm |

### 2. Test Method

### 2.1. Dose Design

2.1.1. In the original prescription, a recommended clinical dosage for adult is 7.6 g crude drug/60 kg/d; in the test, based on the BSA conversion of human and animal, a unit dosage for mouse is 11 times the unit dosage for human, an administered dosage for mouse was (7.6/60)×11=1.4 g crude drug/kg/d, a concentration of test drug liquid was 1 g crude drug/ml, the administered dosage for mouse was converted to a body volume of 1.4/1=1.4 ml/kg/d, thus a high dosage of the original prescription was 2.8 ml/kg/d (equivalent to two times a clinical dosage for human) and a low dosage of the original prescription was 1.4 ml/kg/d (equivalent to the clinical dosage for human).

In the present prescription, a recommended clinical dosage for adult is 7.0 g crude drug/60 kg/d; in the test, based on the BSA conversion of human and animal, a unit dosage for mouse is 11 times the unit dosage for human, an administered dosage for mouse was (7.0/60)×11=1.3 g crude drug/kg/d, a concentration of test drug liquid was 1 g crude drug/ml, the administered dosage for mouse was converted to volume of 1.3/1=1.3 ml/kg/d, thus a high dosage of the present prescription was 2.6 ml/kg/d (equivalent to two times a clinical dosage for human) and a low dosage of the present prescription was 1.3 ml/kg/d (equivalent to the clinical dosage for human).

2.1.2. In the cyclophosphamide for injection: a recommended clinical dosage for adult is 1000 mg/m²/dose, the BSA for adult is 1.6246 m²/60 kg, and a recommended clinical dosage for adult after conversion is 1624.6 mg/60 kg; in the test, based on the BSA conversion of human and animal, a unit dosage for mouse was 11 times the unit dosage for human, an administered dosage for mouse was (1624.6/60)×11=297.84 mg/kg/dose=0.3 g/kg/dose.

### 2.2. Culture of Human Lung Adenocarcinoma Cell Line (A549)

The cells were cultured in a complete medium (DMEM culture solution+10% fetal bovine serum, pH=7.2) and placed in a 37° C. and 5% CO₂ constant-temperature incubator. The culture medium was changed daily, and cells were cultured until a fusion rate of 80%-90%. The cells were digested with 0.25% trypsin, centrifuged, and then passaged. After the cells were cultured to a certain quantity, the cells were diluted with PBS to form a tumor cell suspension solution, and the tumor cell suspension solution was inoculated into a test animal.

### 2.3. Construction of A549 Tumor-Bearing Nude Mouse

### 1) Construction of Primary A549 Tumor-Bearing Nude Mouse

Normally cultured A549 cells were digested with trypsin-EDTA, then centrifuged at 1000 rpm, and resuspended in DMEM (high glucose) after supernatant was discarded, so as to achieve a cell concentration of 10⁶-10⁷ cells/ml. Right axilla of each of three BALB/c nude mice was subcutaneously inoculated with 0.4 ml A549 cell suspension solution. The nude mice inoculated were housed normally, and living conditions and tumor growth of the nude mice were observed.

### 2) Construction of Passage A549 Tumor-Bearing Nude Mouse

After tumors of the primary A549 tumor-bearing nude mice grew to a diameter of about 1 cm, one nude mouse with a good living condition and good tumor growth was selected. The tumors of the nude mouse were peeled off and placed in a glass homogenizer under a sterile environment, 1.5 ml of sterile saline solution was added to prepare homogenate, and the homogenate was then filtered through a 70 μm cell sieve, and filtered homogenate was kept. Right axilla of each of three BALB/c nude mice was subcutaneously inoculated with 0.4 ml the filtered homogenate. The nude mice inoculated were housed normally, and living conditions and tumor growth of the nude mice were observed.

### 2.4. Preparation of Human Lung Cancer Xenograft Model

88 nude mice (half male and half female) weighing 18-20 g were adaptively housed in an experimental animal center for 5 days. When tumors of the passage tumor-bearing nude mice grew to a diameter of about 1.5 cm, three nude mice with a good living condition and good tumor growth were selected. The tumors of the three nude mice were peeled off under a sterile environment, and 3.0 g of the tumors was weighed and placed in a glass homogenizer. 20 ml of sterile saline solution was added to prepare homogenate, then the homogenate was filtered through a 70 μm cell sieve, and filtered homogenate was kept. 8 nude mice were randomly selected as a blank control group, and right axilla of each of remaining 80 nude mice were subcutaneously inoculated with 0.2 ml of the filtered homogenate. The 80 nude mice inoculated were randomly divided into 10 groups according to body weight: model control group, cyclophosphamide group (0.3 g/kg), original-prescription high-dosage group (2.8 g/kg), original-prescription low-dosage group (1.4 g/kg), present-prescription high-dosage group (2.6 g/kg), present-prescription low-dosage group (1.3 g/kg), cyclophosphamide (0.3 g/kg)+original-prescription high-dosage group (2.8 g/kg), cyclophosphamide (0.3 g/kg)+original-prescription low-dosage group (1.4 g/kg), cyclophosphamide (0.3 g/kg)+present-prescription high-dosage group (2.6 g/kg), cyclophosphamide (0.3 g/kg)+present-prescription low-dosage group (1.3 g/kg), where 8 mice were in each group.

### 2.5. Administration

Administration was performed from the second day after inoculation, 0.2 ml/10 g was intragastrically administered, and distilled water was given to the blank control group and the model control group under the same condition. 0.1 ml/10 g of cyclophosphamide was administered in intraperitoneal injection when tumors grew to about 50 mm³.

### 2.6. Sampling

On evening of last administration, the mice were fasted. On next morning, body weights of the mice were measured and the longest and shortest diameters of the tumors were measured. Then, eyeballs of the mice were enucleated, and whole blood (anticoagulation) and serum were collected. Tumors, livers, hearts, spleens, lungs, and kidneys were harvested, and weights were measured in the order of liver, heart, spleen, lung, kidney, and tumor.

### 2.7. Indicator Observation

### 1) Weight

Body weights of the mice were measured once a week and administration dosages of the mice were adjusted accordingly based on the body weights.

### 2) Tumor Volume

Longest diameters (a) and shortest diameters (b) of the tumors were measured with a digital vernier caliper when tumor growth can be seen. Tumor volume was measured, where a formula for calculating the tumor volume (TV) was TV=(1/2) ab², and the tumor volume was measured twice a week.

### 3) Tumor Inhibition Rate and Visceral Organ Index

Tumors, livers, hearts, spleens, lungs, and kidneys were harvested, and weights thereof were measured in the order of liver, heart, spleen, lung, kidney, and tumor. Each tumor inhibition rate and each visceral organ index were calculated by the following formulas:

The each tumor inhibition rate (%)=(average tumor weight of the model control group-average tumor weight of each administration group)/the average tumor weight of the model control×100%; and,

$$The each visceral organ index ( \% ) = ( weight of each visceral organ ) / body weight \times 100 \% .$$

Detection of hemogram indicator: whole blood was used to perform blood cell detection, and serum was detected for glutamic-pyruvic transaminase (GLT), glutathione S-transferase (GST), total bilirubin (TBil), total protein (TP), blood urea nitrogen (BUN), creatinine (CRE), and urea (UA).

### 2.8. Effect Evaluation of Drug Combination

Bürgi formula (q=E (a+b)/(Ea+Eb−Ea×Eb)) was used to evaluate whether the combination of cyclophosphamide and the original prescription or the present prescription has a synergistic or antagonistic effect. E(a+b) represents an effective rate of drug combination, Ea and Eb represent different effective rates of drug alone, q<0.85 means two drugs antagonize each other, q>1.15 means the two drugs synergize, and 0.85<q<1.15 means effects of the two drugs are simply additive.

### 2.9. Statistical Analysis

All data were expressed as average #standard deviation (x̅±SD), results comparison was tested by using T-test, and p<0.05 is considered statistically significant.

### 3. Test Result

3.1. Effect on Tumor Growth of Nude Mouse Inoculated with Human Lung Adenocarcinoma Cell Line (A549)

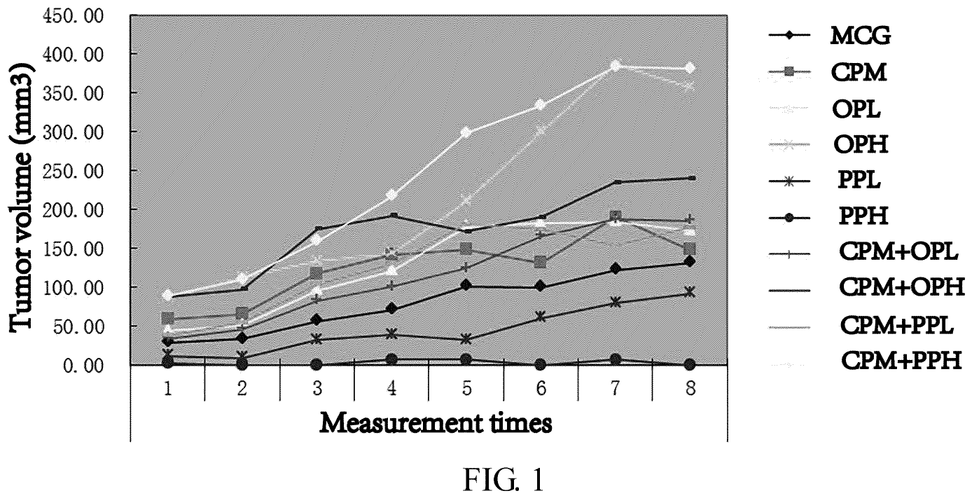

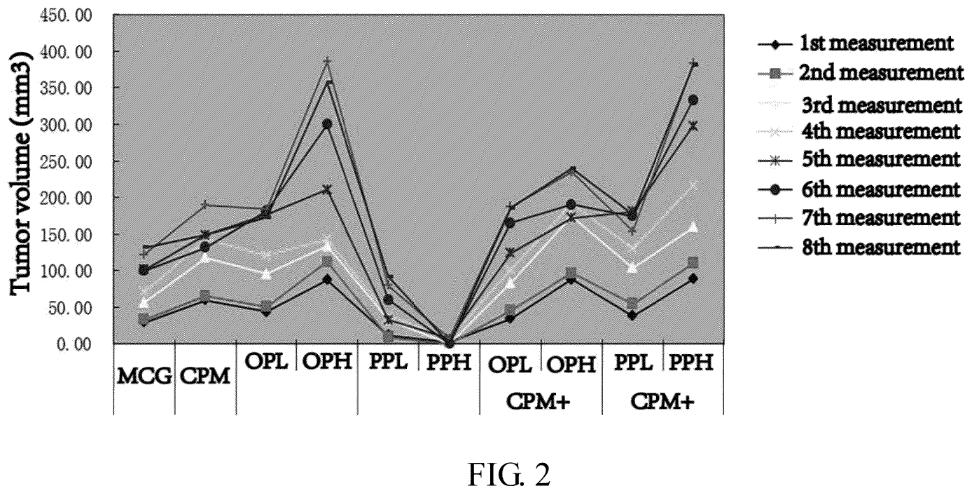

Table 1 and FIGS. 1 and 2 show that after the nude mice were inoculated with human lung adenocarcinoma cells (A549), prophylactic administration began on the second day. From the 27th day of the administration (first measurement) to the 50th day of the administration, the present-prescription low-dosage group and the present-prescription high-dosage group showed a significant inhibition effect on tumor growth of A549 tumor-bearing nude mice, with significant differences (p<0.05, p<0.01) compared to the model control group, where the present-prescription high-dosage group had a tumor inhibition rate of 90.62%.

After drug combination of the original prescription and cyclophosphamide, and drug combination of the present prescription and cyclophosphamide, q was calculated using the Bürgi formula, and q showed that a therapeutic effect of drug combination of present-prescription low dosage and cyclophosphamide (q=0.97) on A549 tumor was the additive effect of the present-prescription low dosage and the cyclophosphamideon, and therapeutic effects of drug combination of original-prescription low dosage and cyclophosphamide (q=0.27), drug combination of original-prescription high dosage and cyclophosphamide (q=0.34), and drug combination of present-prescription high dosage and cyclophosphamide (q=−3.47) on A549 tumor were all mutually antagonistic.

The low-dosage group and the high-dosage group of the original prescription alone, and in combination of cyclophosphamide did not show any preventive effects on the occurrence of human lung adenocarcinoma.

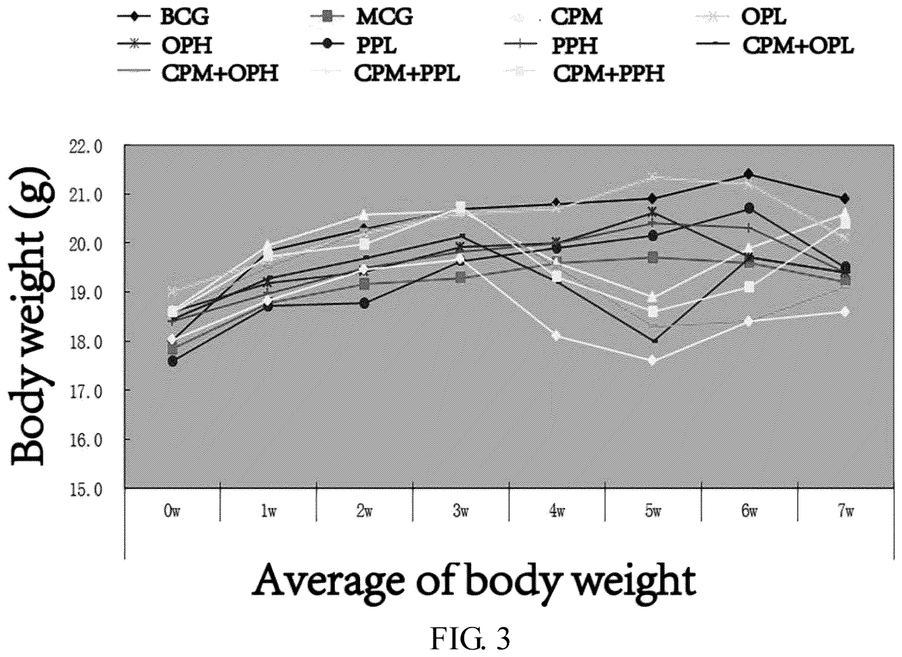

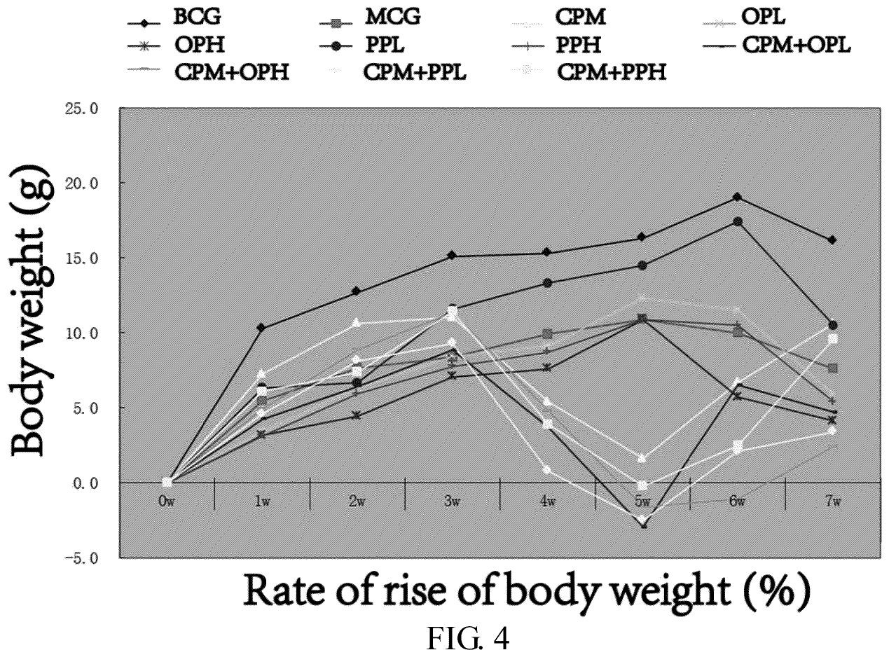

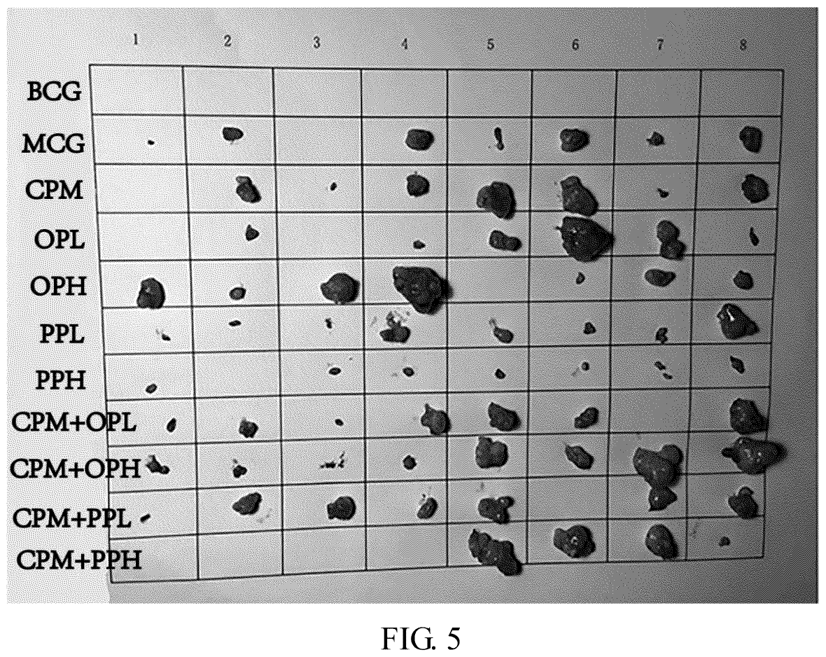

FIGS. 3, 4, and 5 show that during administration, mouse body weights in the original-prescription low-dosage group, the original-prescription high-dosage group, the present-prescription low-dosage group, and the present-prescription high-dosage group increased significantly compared to mouse body weight of the model control group; after combined administration with cyclophosphamide, mouse body weight of each group of the original-prescription low-dosage group, the original-prescription high-dosage group, the present-prescription low-dosage group, and the present-prescription high-dosage group decreased significantly after 4 weeks of the combined administration.

TABLE 1
Effects (X ± SD) of the original prescription and the present prescription on tumor volume
change of tumor-bearing nude mouse inoculated with human lung adenocarcinoma cells (A549)

| | | | | | | | | | | | Tumor | |
|---|---|---|---|---|---|---|---|---|---|---|---|---|
| | Dosage | Animal | Tumor volume after administration (mm³) | | | | | | | | inhibition | |
| Group | (g/kg) | quantity | Day 27 | Day 30 | Day 34 | Day 37 | Day 41 | Day 44 | Day 48 | Day 50 | rate (%) | Q |
| MCG | — | 8 | 28.97 ± | 33.13 ± | 56.31 ± | 70.84 ± | 101.38 ± | 99.90 ± | 121.99 ± | 131.66 ± | — | — |
| | | | 9.96 | 17.83 | 34.69 | 42.44 | 47.95 | 60.16 | 104.02 | 104.34 | | |
| CPM | 0.3 | 8 | 59.41 ± | 65.42 ± | 117.52 ± | 142.12 ± | 148.33 ± | 131.63 ± | 189.98 ± | 148.57 ± | −91.69 | — |
| | | | 55.97 | 104.88 | 188.28 | 203.99 | 181.98 | 157.81 | 250.44 | 170.46 | | |
| OPL | 1.4 | 8 | 44.01 ± | 50.93 ± | 95.84 ± | 120.63 ± | 177.17 ± | 181.78 ± | 184.26 ± | 172.92 ± | −83.67 | — |
| | | | 61.05 | 81.12 | 164.57 | 185.91 | 350.15 | 346.50 | 319.33 | 301.93 | | |
| OPH | 2.8 | 8 | 87.64 ± | 112.16 ± | 133.42 ± | 143.04 ± | 210.52 ± | 299.66 ± | 385.58 ± | 357.20 ± | −206.61 | — |
| | | | 143.11 | 170.79 | 189.52 | 206.99 | 335.94 | 401.44 | 432.73 | 447.41 | | |
| PPL | 1.3 | 8 | 11.62 ± | 9.07 ± | 32.09 ± | 38.87 ± | 32.08 ± | 60.09 ± | 69.66 ± | 91.65 ± | 26.21 | — |
| | | | 13.85* | 16.92* | 59.53 | 55.50 | 48.72* | 90.78 | 98.21 | 107.95 | | |
| PPH | 2.6 | 8 | 2.29 ± | 0.00 ± | 0.00 ± | 7.44 ± | 7.36 ± | 0.00 ± | 7.30 ± | 0.00 ± | 90.62 | — |
| | | | 6.47** | 0.00** | 0.00** | 21.04** | 20.82** | 0.00** | 20.64** | 0.00** | | |
| CPM + | 0.3 + | 8 | 34.30 ± | 45.66 ± | 82.61 ± | 100.58 ± | 123.88 ± | 165.35 ± | 187.02 ± | 185.52 ± | −67.12 | 0.27 |
| OPL | 1.4 | | 31.59 | 61.03 | 104.47 | 108.50 | 161.19 | 169.58 | 216.56 | 228.77 | | |
| CPM + | 0.3 + | 8 | 88.21 ± | 97.00 ± | 17480 ± | 191.73 ± | 171.85 ± | 190.12 ± | 234.61 ± | 239.68 ± | −164.48 | 0.34 |
| OPH | 2.8 | | 128.13 | 129.78 | 262.85 | 225.75 | 224.11 | 260.96 | 329.25 | 306.39 | | |
| CPM + | 0.3 + | 8 | 38.29 ± | 54.74 ± | 103.86 ± | 130.09 ± | 180.62 ± | 174.35 ± | 153.52 ± | 177.05 ± | −40.14 | 0.97 |
| PPL | 1.3 | | 50.12 | 55.00 | 80.20 | 81.59 | 141.94 | 141.01 | 133.89 | 149.60 | | |
| CPM + | 0.3 + | 8 | 88.81 ± | 110.03 ± | 159.26 ± | 216.93 ± | 297.87 ± | 333.07 ± | 383.10 ± | 380.78 ± | −284.24 | 3.47 |
| PPH | 2.6 | | 118.12 | 143.62 | 192.93 | 266.70 | 413.73 | 409.54 | 440.69 | 394.11 | | |

Remark:
*represents p < 0.05 when compared with the model control group, and
**represents p < 0.01 when compared with the model control group;
MCG represents the model control group, CPM represents the cyclophosphamide group, OPH represents the original-prescription high-dosage group, OPL represents the original-prescription low-dosage group, PPH represents the present-prescription high-dosage group, PPL represents the present-prescription low-dosage group, CPM + OPH represents the cyclophosphamide + original-prescription high-dosage group, CPM + OPL represents the cyclophosphamide + original-prescription low-dosage group, CPM + PPH represents the cyclophosphamide + present-prescription high-dosage group, and CPM + PPL represents the cyclophosphamide + present-prescription low-dosage group.

In the present tables and figures of the present disclosure, prescription lacking Sophora flavescens is the present prescription.

3.2. Effect on Liver Function of Nude Mice with Human Lung Adenocarcinoma Cells (A549)

Table 2 shows that there were no significant changes in liver index, serum ALT content, serum AST content, ALT/AST content, serum TBil content, and serum TP content in the model control group, the cyclophosphamide group, and each administration group.

TABLE 2
Effects (X ± SD) of the original prescription and the
present prescription on liver function of tumor-bearing nude
mouse inoculated with human lung adenocarcinoma cells (A549)

| | | | | Serum | Serum | | Serum | Serum |
|---|---|---|---|---|---|---|---|---|
| | Dosage | Animal | Liver | ALT | AST | ALT/ | TBil | TP |
| Group | (g/kg) | quantity | index | (UI/L) | (UI/L) | AST | (μmol/L) | (g/L) |
| BCG | — | 8 | 0.0440 ± | 36.3 ± | 128.4 ± | 3.540 ± | 1.60 ± | 57.14 ± |
| | | | 0.0065 | 3.7 | 16.1 | 0.247 | 0.50 | 2.87 |
| MCG | — | 8 | 0.0462 ± | 38.6 ± | 149.9 ± | 3.909 ± | 1.98 ± | 54.72 ± |
| | | | 0.0037 | 4.7 | 16.6 | 0.438 | 0.43 | 2.06 |
| CPM | 0.3 | 8 | 0.0481 ± | 40.2 ± | 137.0 ± | 3.505 ± | 1.59 ± | 51.06 ± |
| | | | 0.0027 | 8.2 | 6.1 | 0.562 | 0.45 | 1.32 |
| OPL | 1.4 | 8 | 0.0457 ± | 40.6 ± | 156.3 ± | 3.845 ± | 2.00 ± | 57.59 ± |
| | | | 0.0050 | 3.5 | 29.7 | 0.611 | 0.42 | 3.48 |
| OPH | 2.8 | 8 | 0.0440 ± | 40.9 ± | 154.5 ± | 3.853 ± | 1.94 ± | 55.26 ± |
| | | | 0.0028 | 7.1 | 27.8 | 0.298 | 0.29 | 2.57 |
| PPL | 1.3 | 8 | 0.0430 ± | 37.8 ± | 175.7 ± | 4.431 ± | 2.01 ± | 56.21 ± |
| | | | 0.0016 | 7.6 | 101.8 | 1.410 | 0.73 | 3.07 |
| PPH | 2.6 | 8 | 0.0444 ± | 38.2 ± | 143.1 ± | 3.732 ± | 1.76 ± | 56.41 ± |
| | | | 0.0032 | 2.7 | 28.1 | 0.576 | 0.54 | 3.78 |
| CPM + | 0.3 + 1.4 | 8 | 0.0473 ± | 108.2 ± | 264.9 ± | 2.940 ± | 2.01 ± | 52.17 ± |
| OPL | | | 0.0037 | 143.3 | 296.3 | 0.735 | 0.22 | 2.42 |
| CPM + | 0.3 + 2.8 | 8 | 0.0545 ± | 38.7 ± | 145.5 ± | 3.778 ± | 1.76 ± | 52.33 ± |
| OPH | | | 0.0055 | 7.1 | 29.0 | 0.568 | 0.44 | 2.54 |
| CPM + | 0.3 + 1.3 | 8 | 0.0500 ± | 39.3 ± | 137.2 ± | 3.672 ± | 1.42 ± | 51.80 ± |
| PPL | | | 0.0037 | 14.0 | 22.2 | 0.749 | 0.59 | 2.96 |
| CPM + | 0.3 + 2.6 | 8 | 0.0477 ± | 36.7 ± | 134.2 ± | 3.703 ± | 2.15 ± | 51.04 ± |
| PPH | | | 0.0024 | 5.6 | 21.6 | 0.704 | 0.32 | 2.58 |

Remark:
BCG represents the blank control group.

3.3. Effect on Kidney Function of Nude Mouse Inoculated with Human Lung Adenocarcinoma Cells (A549)

Table 3 shows that there were no significant changes in kidney index, serum BUN content, serum UA content, and serum CRE content in the model control group, the cyclophosphamide group, and each administration group.

TABLE 3
Effects (X ± SD) of the original prescription and the
present prescription on kidney function of tumor-bearing nude
mouse inoculated with human lung adenocarcinoma cells (A549)

| | | | | Blood urea | | Creatinine |
|---|---|---|---|---|---|---|
| | Dosage | Animal | Kidney | nitrogen (BUN) | Urea (UA) | (CRE) |
| Group | (g/kg) | quantity | index | (mmol/L) | (μmol/L) | (mg/dL) |
| BCG | — | 8 | 0.0150 ± | 7.50 ± | 239.51 ± | 6.90 ± |
| | | | 0.0016 | 1.00 | 73.60 | 1.10 |
| MCG | — | 8 | 0.0151 ± | 8.50 ± | 173.08 ± | 5.90 ± |
| | | | 0.0010 | 1.00 | 71.54 | 1.60 |
| CPM | 0.3 | 8 | 0.0151 ± | 6.90 ± | 187.56 ± | 4.90 ± |
| | | | 0.0017 | 0.50 | 40.46 | 0.90 |
| OPL | 1.4 | 8 | 0.0156 ± | 8.20 ± | 198.57 ± | 8.00 ± |
| | | | 0.0029 | 1.50 | 76.10 | 5.10 |
| OPH | 2.8 | 8 | 0.0144 ± | 7.50 ± | 258.66 ± | 6.70 ± |
| | | | 0.0013 | 0.40 | 27.07 | 1.00 |
| PPL | 1.3 | 8 | 0.0154 ± | 8.90 ± | 236.63 ± | 6.50 ± |
| | | | 0.0031 | 1.30 | 104.00 | 1.80 |
| PPH | 2.6 | 8 | 0.0158 ± | 8.20 ± | 178.49 ± | 6.10 ± |
| | | | 0.0021 | 0.80 | 37.67 | 1.80 |
| CPM + | 0.3 + 1.4 | 8 | 0.0146 ± | 7.40 ± | 208.36 ± | 5.20 ± |
| OPL | | | 0.0009 | 0.50 | 51.90 | 0.90 |
| CPM + | 0.3 + 2.8 | 8 | 0.0150 ± | 7.20 ± | 196.78 ± | 6.00 ± |
| OPH | | | 0.0012 | 1.30 | 38.18 | 2.60 |
| CPM + | 0.3 + 1.3 | 8 | 0.0147 ± | 7.40 ± | 189.29 ± | 5.10 ± |
| PPL | | | 0.0012 | 1.00 | 37.44 | 1.20 |
| CPM + | 0.3 + 2.6 | 8 | 0.0194 ± | 7.70 ± | 248.34 ± | 4.80 ± |
| PPH | | | 0.0045 | 1.80 | 47.38 | 1.00 |

3.4. Effects on Heart, Spleen, and Lung Indices of Nude Mouse Inoculated with Human Lung Adenocarcinoma Cells (A549)

Table 4 shows that: 1) compared with the blank control group, the heart index of the model control group showed an increasing trend; heart indices of the original-prescription high-dosage group, the present-prescription low-dosage group, the present-prescription high-dosage group, the cyclophosphamide+original-prescription low-dosage group, the cyclophosphamide+original-prescription high-dosage group were significantly decreased and had significant differences (p<0.05, p<0.01) compared with the model control group and the cyclophosphamide group; 2) there was no statistical significance in the spleen index and lung index of the model control group, the cyclophosphamide group, and each administration group.

TABLE 4
Effects (X ± SD) of the original prescription and the present
prescription on heart index, spleen index, and lung index of tumor-
bearing nude mouse inoculated with human lung adenocarcinoma cells (A549)

| | Dosage | Animal | | | |
|---|---|---|---|---|---|
| Group | (g/kg) | quantity | Heart index (%) | Spleen index (%) | Lung index (%) |
| BCG | — | 8 | 0.57 ± 0.06 | 7.50 ± 1.00 | 239.51 ± 73.60 |
| MCG | — | 8 | 0.62 ± 0.08 | 8.50 ± 1.00 | 173.08 ± 71.54 |
| CPM | 0.3 | 8 | 0.62 ± 0.07 | 6.9 ± 0.5 | 187.56 ± 40.46 |
| OPL | 1.4 | 8 | 0.59 ± 0.04 | 8.20 ± 1.50 | 198.57 ± 76.10 |
| OPH | 2.8 | 8 | 0.54 ± 0.03^{#Δ} | 7.50 ± 0.40 | 258.66 ± 27.07 |
| PPL | 1.3 | 8 | 0.52 ± 0.04^{#ΔΔ} | 8.90 ± 1.30 | 236.63 ± 104.00 |
| PPH | 2.6 | 8 | 0.54 ± 0.04^{#Δ} | 8.20 ± 0.80 | 178.49 ± 37.67 |
| CPM + | 0.3 ± 1.4 | 8 | 0.54 ± 0.03^{#Δ} | 7.40 ± 0.50 | 208.36 ± 51.90 |
| OPL | | | | | |
| CPM + | 0.3 ± 2.8 | 8 | 0.53 ± 0.06^{#Δ} | 7.20 ± 1.30 | 196.78 ± 38.18 |
| OPH | | | | | |
| CPM + | 0.3 ± 1.3 | 8 | 0.58 ± 0.03 | 7.40 ± 1.00 | 189.29 ± 37.44 |
| PPL | | | | | |
| CPM + | 0.3 ± 2.6 | 8 | 0.59 ± 0.06 | 7.70 ± 1.80 | 248.34 ± 47.38 |
| PPH | | | | | |

Remark:
* represents p < 0.05 when compared with the blank control group,
** represents p < 0.01 when compared with the blank control group,
^{#}represents p < 0.05 when compared with the model control group,
^{##}represents p < 0.01 when compared with the model control group,
^{Δ}represents p < 0.05 when compared with the cyclophosphamide group, and
^{ΔΔ}represents p < 0.01 when compared with the cyclophosphamide group.

3.5. Effect on Blood Routine of Nude Mouse Inoculated with Human Lung Adenocarcinoma Cells (A549)

Table 5 shows that the total numbers of white blood cells, neutrophils, lymphocytes, and monocytes in the model control group were significantly decreased, and had significant differences (p<0.05, p<0.01) compared to the blank control group.

The original-prescription high-dosage group, the present-prescription low-dosage, and the present-prescription high-dosage group, and drug combination of cyclophosphamide and the original-prescription low dosage, cyclophosphamide and the original-prescription high dosage, cyclophosphamide with the present-prescription low dosage, and cyclophosphamide with the present-prescription high dosage, all showed increased total numbers of the white blood cells, the neutrophils, the lymphocytes, and the monocytes, with significant differences (p<0.05, p<0.01) compared to the model control group.

Effects of drug combination of cyclophosphamide and the original-prescription high dosage and drug combination of cyclophosphamide and the present-prescription low dosage on increasing the number of white blood cells, neutrophils, lymphocytes, and monocytes than the effect of each drug individual, with significant differences (p<0.05, p<0.01) compared with the cyclophosphamide group.

The basophils in the model control group were significantly increased; the cyclophosphamide group, the present prescription, and the original prescription, as well as drug combination of the present prescription and cyclophosphamide, and drug combination of the original prescription and cyclophosphamide, all caused significantly increased basophils.

Contents of red blood cell, platelet, hemoglobin, and eosinophils in the blank control group, the model control group, and each administration group fluctuated within a normal range.

TABLE 5
Effects (X ± SD) of the original prescription and the present prescription on liver
function of tumor-bearing nude mouse inoculated with human lung adenocarcinoma cells (A549)

| | | | Red | | | White | | | | | |
|---|---|---|---|---|---|---|---|---|---|---|---|
| | | | blood | | Hemo- | blood | Neutro- | Lympho- | Mono- | Baso- | Eosi- |
| | | | cell | Platelet | globin | cell | phil | cyte | cyte | phil | nophil |
| | Dosage | Animal | (RBC) | (PLT) | (HGB) | (WBC) | (NEUT) | (LYMP) | (MONO) | (BASO) | (EO) |
| Group | (g/kg) | quantity | (10¹²/L) | (10⁹/L) | (10⁹/L) | (10⁹/L) | (10⁹/L) | (10⁹/L) | (10⁹/L) | (10⁹/L) | (10⁹/L) |
| BCG | — | 8 | 10.59 ± | 865.00 ± | 165.75 ± | 2.50 ± | 0.13 ± | 1.63 ± | 0.08 ± | 0.71 ± | 0.00 ± |
| | | | 0.28 | 60.10 | 4.74 | 0.30 | 0.03 | 0.35 | 0.05 | 0.23 | 0.00 |
| MCG | — | 8 | 10.59 ± | 860.57 ± | 165.00 ± | 1.90 ± | 0.06 ± | 0.70 ± | 0.04 ± | 1.07 ± | 0.00 ± |
| | | | 0.40 | 100.84 | 7.66 | 0.50** | 0.03* | 0.32** | 0.03* | 0.55 | 0.00 |
| CPM | 0.3 | 8 | 10.40 ± | 1079.00 ± | 162.50 ± | 5.1 ± | 0.34 ± | 0.98 ± | 0.40 ± | 3.33 ± | 0.00 ± |
| | | | 0.39 | 224.6 | 5.50 | 2.7^{##} | 0.12^{##} | 0.69 | 0.25^{##} | 1.73^{##} | 0.00 |
| OPL | 1.4 | 8 | 10.90 ± | 870.75 ± | 168.63 ± | 1.90 ± | 0.08 ± | 0.80 ± | 0.05 ± | 0.99 ± | 0.00 ± |
| | | | 0.48 | 251.00 | 8.35 | 0.60 | 0.04 | 0.41 | 0.02 | 0.47 | 0.00 |
| OPH | 2.8 | 8 | 11.12 ± | 765.29 ± | 171.43 ± | 3.50 ± | 0.10 ± | 1.82 ± | 0.15 ± | 1.49 ± | 0.00 ± |
| | | | 0.3 | 327.04 | 4.96 | 1.20^{##} | 0.04^{ΔΔ} | 1.12^{#} | 0.09^{#Δ} | 0.79 | 0.00 |
| PPL | 1.3 | 8 | 11.02 ± | 908.50 ± | 172.88 ± | 4.50 ± | 0.13 ± | 2.91 ± | 0.26 ± | 1.15 ± | 0.00 ± |
| | | | 0.31 | 66.67 | 5.11 | 1.30^{##} | 0.07^{#ΔΔ} | 1.18^{##ΔΔ} | 0.29 | 0.38 | 0.00 |
| PPH | 2.6 | 8 | 11.06 ± | 836.50 ± | 172.75 ± | 4.00 ± | 0.13 ± | 2.90 ± | 0.10 ± | 0.91 ± | 0.00 ± |
| | | | 0.30 | 283.94 | 4.53 | 1.50^{##} | 0.06^{#ΔΔ} | 1.18^{##ΔΔ} | 0.06^{#ΔΔ} | 0.27 | 0.00 |
| CPM + | 0.3 + 1.4 | 8 | 10.51 ± | 958.43 ± | 166.00 ± | 5.06 ± | 0.21 ± | 1.50 ± | 0.23 ± | 3.63 ± | 0.00 ± |
| OPL | | | 0.56 | 80.82 | 8.83 | 2.10^{##} | 0.09^{##ΔΔ} | 0.79^{#} | 0.20^{#} | 1.26^{##} | 0.00 |
| CPM + | 0.3 + 2.8 | 8 | 10.91 ± | 1087.38 ± | 169.13 ± | 12.20 ± | 0.56 ± | 2.93 ± | 0.94 ± | 7.75 ± | 0.00 ± |
| OPH | | | 0.61 | 392.63 | 9.58 | 9.00^{##} | 0.04^{##} | 3.63 | 0.68^{##Δ} | 5.12^{##Δ} | 0.00 |
| CPM + | 0.3 + 1.3 | 8 | 11.16 ± | 659.357 ± | 172.00 ± | 9.10 ± | 0.45 ± | 2.23 ± | 0.60 ± | 5.78 ± | 0.00 ± |
| PPL | | | 0.65 | 263.86 | 10.69 | 2.10^{##} | 0.13^{##} | 0.62^{##ΔΔ} | 0.35^{##} | 1.82^{##Δ} | 0.00 |
| CPM + | 0.3 + 2.6 | 8 | 11.02 ± | 742.20 ± | 171.80 ± | 5.00 ± | 0.24 ± | 1.10 ± | 0.23 ± | 3.44 ± | 0.01 ± |
| PPH | | | 0.49 | 196.47 | 8.17 | 2.20^{##} | 0.15^{##} | 0.27^{#} | 0.14^{##} | 1.85^{##} | 0.01 |

Remark:
*represents p < 0.05 when compared with the blank control group,
**represents p < 0.01 when compared with the blank control group,
^{#}represents p < 0.05 when compared with the model control group,
^{##}represents p < 0.01 when compared with the model control group,
^{Δ}represents p < 0.05 when compared with the cyclophosphamide group, and
^{ΔΔ}represents p < 0.01 when compared with the cyclophosphamide group.

### 4. Conclusion

1) The present-prescription low dosage and the present-prescription high dosage have significant inhibitory effects on tumor growth of A-549 tumor-bearing nude mouse, where the present-prescription high dosage has a tumor inhibition rate of 90.62%.

2) Drug combination of the present-prescription low dosage and cyclophosphamide has a simple additive effect (q=0.97) on treatment of A549 tumor.

3) The original-prescription low dosage, the original-prescription high dosage, the present-prescription low dosage, and the present-prescription high dosage have significant increase effects on mouse body weight.

4) The original-prescription high dosage, the present-prescription low dosage, the present-prescription high dosage, drug combination of the original-prescription low dosage and cyclophosphamide, and drug combination of the original-prescription high dosage and cyclophosphamide, can significantly reduce heart index.

5) The original-prescription high dosage, the present-prescription low dosage, the present-prescription high dosage, drug combination of the original-prescription low dosage and cyclophosphamide, drug combination of the original-prescription high dosage and cyclophosphamide, drug combination of the present-prescription low dosage and cyclophosphamide, and drug combination of the present-prescription high dosage and cyclophosphamide, all can increase the numbers of white blood cells, neutrophils, lymphocytes, and monocytes.

6) Drug combination of the original-prescription high dosage and cyclophosphamide, and drug combination of the present-prescription low dosage and cyclophosphamide, both have better effects on increasing the numbers of white blood cells, neutrophils, lymphocytes, and monocytes than each drug alone.

7) The original prescription, the present prescription, drug combination of the original prescription and cyclophosphamide, and drug combination of the present prescription and cyclophosphamide, all can significantly increase the number of Basophils.

8) There are no significant changes in liver index, kidney index, spleen index, lung index, serum BUN content, serum UA content, and serum CRE content in each drug alone group and each drug combination group, and serum ALT content, serum AST content, ALT/AST content, serum TBil content, and serum TP content are not affected.

9) Contents of red blood cells, platelets, hemoglobin, and eosinophils in each drug alone group and each drug combination group fluctuate within a normal range.

Prescription analysis for the traditional Chinese medicine composition of the present disclosure is as follows.

### Ingredient:

Ganoderma lucidum (stemless Ganoderma lucidum and phellinuspini), Astragalus membranaceus;
Atractylodes lancea, Glycyrrhiza uralensis, Cinnamomum cassia, Aucklandia radix, Wolfiporia cocos, Syzygium aromaticum;
Lonicera japonica, Dendranthema morifolium; and,
Mentha haplocalyx, Ligusticum chuanxiong.

King: Ganoderma lucidum (stemless Ganoderma lucidum and phellinuspini) and Astragalus membranaceus.

Minister: Atractylodes lancea, Glycyrrhiza uralensis, Cinnamomum cassia, Aucklandia radix, Wolfiporia cocos, and Syzygium aromaticum.

Assistant: Lonicera japonica and Dendranthema morifolium.

Messenger: Ligusticum chuanxiong and Mentha haplocalyx.

Indication: Tumor patients with Qi deficiency of spleen and lung, Qi and blood stasis, phlegm-dampness and toxin accumulation, and weakened immunity.

Function: Tonifying spleen and building lung, dispersing lump and resolving accumulation, increasing immune cell such as white blood cell, neutrophil, lymphocyte, etc. and enhancing immunity.

Analysis: Ganoderma lucidum tonifies heart, liver, spleen, lung, and kidney, invigorates spleen and replenishes Qi, and enhances body immunity; Astragalus membranaceus invigorates spleen and nourishes lung, benefits qi for ascending Yang energy, and enhances body immunity; Ganoderma lucidum and Astragalus membranaceus can inhibit growth of tumor telomerase; Atractylodes lancea, Wolfiporia cocos, and Glycyrrhiza uralensis invigorate spleen and eliminate dampness; Cinnamomum cassia, Aucklandia radix, Syzygium aromaticum are pungent and warm, and can regulate Qi, warm spleen and stomach, dispel coldness, and relieve pain; Lonicera japonica clears heat-toxin; Dendranthema morifolium clears liver, improves eyesight and removes toxicity; Mentha haplocalyx is pungent and cold and removes toxicity, disperses stagnated liver Qi for relieving Qi stagnation; Ligusticum chuanxiong regulates Qi, promotes blood circulation, and relieves pain.

In the present prescription, Ganoderma lucidum and Astragalus membranaceus are as King material to invigorate spleen and replenish Qi, tonify heart, liver, spleen, lung, and kidney, strengthen body resistance and consolidate body vitality, and resist tumor. Atractylodes lancea, Wolfiporia cocos, and Glycyrrhiza uralensis are as Minister material to invigorate spleen and eliminate dampness, and Cinnamomum cassia, Aucklandia radix, and Syzygium aromaticum are as Minister material to regulate Qi and warm spleen and stomach, dispel coldness and relieve pain. Dendranthema morifolium and Lonicera japonica are as Assistant material to clear heat-toxin. Ligusticum chuanxiong and Mentha haplocalyx are as Messenger material to clear liver, promote blood circulation, and relieve pain.

The above is only used to illustrate the technical solutions of the present disclosure and not to limit the present disclosure. Any other modifications or equivalent substitutions made by those ordinary skilled in the art to the technical solutions of the present disclosure, should be included in the protection scope of the claims of the present disclosure as long as they do not depart from the spirit and scope of the technical solutions of the present disclosure.

What is claimed is:

1. A traditional Chinese medicine composition for treatment of lung tumor, comprising the following raw materials by weight: 5-15 parts of Dendranthema morifolium, 2-12 parts of Lonicera japonica, 5-15 parts of Mentha haplocalyx, 5-15 parts of Wolfiporia cocos, 10-20 parts of Atractylodes lancea, 0.5-3 parts of Cinnamomum cassia, 0.5-3 parts of Syzygium aromaticum, 3-12 parts of Astragalus membranaceus, 5-15 parts of Ganoderma lucidum, 10-20 parts of Ligusticum chuanxiong, 5-12 parts of Aucklandia radix, and 2-10 parts of Glycyrrhiza uralensis.

2. The traditional Chinese medicine composition for treatment of lung tumor according to claim 1, wherein the composition comprises the following raw materials by weight: 5-10 parts of Dendranthema morifolium, 3-8 parts of Lonicera japonica, 5-10 parts of Mentha haplocalyx, 5-10 parts of Wolfiporia cocos, 12-20 parts of Atractylodes lancea, 0.5-3 parts of Cinnamomum cassia, 0.5-3 parts of Syzygium aromaticum, 5-10 parts of Astragalus membranaceus, 5-10 parts of Ganoderma lucidum, 11-15 parts of Ligusticum chuanxiong, 8-12 parts of Aucklandia radix, and 3-8 parts of Glycyrrhiza uralensis.

3. The traditional Chinese medicine composition for treatment of lung tumor according to claim 1, wherein the composition comprises the following raw materials by weight: 6-9 parts of Dendranthema morifolium, 3-6 parts of Lonicera japonica, 6-9 parts of Mentha haplocalyx, 6-9 parts of Wolfiporia cocos, 15-18 parts of Atractylodes lancea, 1-3 parts of Cinnamomum cassia, 0.5-1.5 parts of Syzygium aromaticum, 6-10 parts of Astragalus membranaceus, 6-10 parts of Ganoderma lucidum, 12-15 parts of Ligusticum chuanxiong, 8-11 parts of Aucklandia radix, and 3-6 parts of Glycyrrhiza uralensis.

4. The traditional Chinese medicine composition for treatment of lung tumor according to claim 1, wherein a dosage form of the composition is injection, honey-refined pill, hydrous pill, capsule, tablet, dripping pill, powder, oral liquid medicine, gel, extract, or film.

5. A preparation method for the traditional Chinese medicine composition for treatment of lung tumor according to claim 1, comprising the following steps:
(1) weighing each of traditional Chinese medicine raw materials in accordance with the weight ratio, separately grinding the raw materials to a particle size of 10-100 mesh, sieving, and then mixing evenly to obtain an evenly-mixed traditional Chinese medicine mixture;
(2) preparing an ethanol solution with a concentration of 40-95% for later use, soaking the evenly-mixed traditional Chinese medicine mixture in the prepared ethanol solution with the concentration of 40-95% for 10-48 hours to obtain a soaked traditional Chinese medicine mixture, adding the soaked traditional Chinese medicine mixture into a percolating bucket in batches, and then gently pressing the soaked traditional Chinese medicine mixture to a flat state;
(3) adding the prepared ethanol solution with the concentration of 40-95% into the percolating bucket until the ethanol solution is 1-2 cm higher than a surface of the soaked traditional Chinese medicine mixture, soaking for a preset duration, then adding remaining ethanol solution into the percolating bucket for percolation to obtain an alcohol solution and medicine herb residue, keeping the medicine herb residue for later use, and concentrating the obtained alcohol solution under reduced pressure and then drying to obtain a traditional Chinese medicine composite powder A; and,
(4) adding water to the medicine herb residue, wherein the amount of the water is 2-10 times the amount of the medicine herb residue, decocting with water and extracting for 0.5-2 hours, centrifuging and filtering to obtain a medicine solution, concentrating the medicine solution under reduced pressure and then drying to obtain a traditional Chinese medicine composite powder B, and mixing the traditional Chinese medicine composite powder A and the traditional Chinese medicine composite powder B evenly to obtain the traditional Chinese medicine composition.

6. The preparation method for the traditional Chinese medicine composition for treatment of lung tumor according to claim 5, wherein in step (1), each of the traditional Chinese medicine raw materials are separately grinded to the particle size of 20-40 mesh; in step (2) and step (3), the concentration of the prepared ethanol solution is 60-80%.

7. The preparation method for the traditional Chinese medicine composition for treatment of lung tumor according to claim 6, wherein in step (2), the evenly-mixed traditional Chinese medicine mixture is soaked for 18-30 hours; in step (3), the preset duration is 2-48 hours.

8. A preparation method for the traditional Chinese medicine composition for treatment of lung tumor according to claim 1, comprising the following steps:
(1) weighing medicinal herbs of Ligusticum chuanxiong, Atractylodes lancea, Aucklandia radix, Syzygium aromaticum, Cinnamomum cassia, and Mentha haplocalyx in accordance with the weight ratio, adding water in moderation to extract volatile oil, adding cyclodextrin for inclusion, and decocting with water and then filtering to obtain filtrate for later use;
(2) mixing medicinal herb residue obtained after extracting the volatile oil with weighed Wolfiporia cocos, Astragalus membranaceus, Dendranthema morifolium, Ganoderma lucidum, Lonicera japonica, and Glycyrrhiza uralensis in accordance with the weight ratio, adding water for decocting, filtering to obtain filtered medicinal herb solution, and combining the filtered medicinal herb solution with the filtrate to obtain a mixed medicine herb solution;
(3) concentrating the mixed medicine herb solution through a water bath to obtain a clear paste, adding ethanol to increase an alcohol content to 50-80% to obtain a medicine alcohol mixture; and,
(4) concentrating the medicine alcohol mixture under reduced pressure, drying, and then adding volatile-oil cyclodextrin inclusion complex to obtain the traditional Chinese medicine composition.

9. The preparation method for the traditional Chinese medicine composition for treatment of lung tumor according to claim 8, wherein in step (1), a volatile-oil extraction solvent is water, the amount of the volatile-oil extraction solvent is 4-15 times the amount the medicinal herbs, an extraction duration for extracting the volatile oil is 2-12 hours, the obtained volatile oil is performed cyclodextrin inclusion at 40° C. or below, and an inclusion duration is 1-10 hours.
